(19) United States
(12) Patent Application Publication
(10) Pub. No.: US 2007/0181122 A1
(43) Pub. Date: Aug. 9, 2007

(54) Intubation positioning, breathing facilitator and non-invasive assist ventilation device

(76) Inventor: Jan Paul Mulier, Brugge (BE)

Correspondence Address: Jan Paul Mulier, veldstraat 14, 8200 Brugge, Brugge, B 8200, BE

(21) Appl. No.: 11/307,388

(22) Filed: Feb. 5, 2006

(51) Int. Cl. A61M 15/00; A61B 5/08; A62B 7/00

(52) U.S. Cl. 128/202.16; 600/529; 128/202.18

(57) ABSTRACT

This invention can be used in three different ways for patients who are lying down in bed or on the operating table. First it facilitates the endotracheal intubation, secondly it facilitates the spontaneous breathing of obese patients and thirdly it assists the spontaneous inspiration and expiration in a non-invasive way. This invention device is positioned under the patient before he is asleep without disturbing him. It allows a gradual elevation of the lower and or upper thorax, a gradual elevation of the head giving a flexion of the neck and a gradual hyperextension of the head. After intubation the position is returned to normal without need for removing the invention device. This invention elevates the spinal column and therefore the thorax is no more compressed and the ribs can move free. Inspiration requires less force and the patient can be breathing easier even when lying down. In this invention the spinal column elevation can also be inflated in a synchronized way with the respiration of the patient. During inspiration the spinal column is elevated, facilitating the inspiration. During expiration the elevation is lowered, facilitating the expiration. The work of breathing is reduced for the patient resulting in larger minute volume ventilation or less oxygen consumption.

### FIELD OF THE INVENTION

The present invention relates to a system and method that facilitates the endotracheal intubation and the spontaneous breathing of patients and that stimulates the spontaneous breathing of patients in a non-invasive way. In most patients only one of the three applications can be used, although some patients might benefit from more than one application.

### SUMMARY OF THE INVENTION

The system and method of this invention can be used in three different ways for patients who are lying down in supine position in bed or on the operating table. First it facilitates the endotracheal intubation, secondly it facilitates the spontaneous breathing of obese patients and thirdly it assists the spontaneous inspiration and expiration in a non-invasive way. The sniffing position is the ideal position to visualize the vocal cords during laryngoscopy for intubation of the trachea with an endotracheal tube. The sniffing position consists of a pillow under the head to elevate the head and to improve the alignment of the tracheal axis, pharyngeal axis and axis of the mouth. However these positions do not guarantee always a good visualization, certainly in the obese patients with a short neck where elevation of the upper thorax can help. Instead of elevating the total upper thorax as described until now a folded blanket put under the spinal column of the thorax elevates only the trachea to anterior and stretches the neck.

More exactly the inventor found that elevation of the thoracic spinal column moves the trachea anterior and elevation of the lower thoracic and abdominal spinal column rotates the tracheal axis more in line with the pharyngeal axis. This position together with an elevation and a hyperextension of the head improves further the alignment of the axes and facilitates the laryngoscopy. In most anesthetic courses of intubation however the opposite is learned, because elevation of the upper thorax rotates the trachea opposite and increases the angle between trachea and pharynx, what makes that no one tried to elevate the lower thorax. Certainly in obese patients this helps. It is however difficult to position it before the patient is asleep, difficult to adapt the thickness and difficult to remove after intubation. This invented device is positioned flat under the patient before he is asleep without disturbing him. It allows a gradual elevation and rotation of the spinal column at the desired level of the thorax, a gradual elevation of the head to flex the neck and a gradual hyperextension of the head created by an extension at the atlanto-axial joint. After intubation the position is returned to normal without need of removing the invented device. The device has extra inflatable compartments to elevate the upper thorax if necessary as during trendelenburg position for preventing the patient to slide of the table. A first longitudinal balloon consisting of two compartments, an upper under the upper thorax and a lower under the lower thorax and abdomen is positioned in the length under the spinal column and allows a gradual elevation by inflating both compartments and tilting of the thorax by inflating the lower compartment more than the upper compartment. A second half circular balloon tube under the upper part of the head stabilizes the head and allows a gradual elevation of the head to achieve the sniffing position and a third half circular balloon under the lower part of the head gives a gradual hyperextension of the head. The four gradual movements allow a better ideal alignment of the tracheal axis, the pharyngeal axis and the mouth axis and facilitate intubation. This is the first way to use this invention.

After the intubation all balloons except the lower under the abdomen are emptied what facilitates central venous puncture if required. During the operation inflation of two triangular balloons under the shoulders, together with an inflation of the head support allow stabilization of the upper thorax and prevention of gliding of the table required in extreme trendelenburg position.

Spontaneous breathing is easiest when staying upright, certainly when people have to speak, to sing or to blow on an instrument. Total lung capacity decline when a person is lying down and this is certainly true for obese patients. Some persons prefer some extra pillows or want to sleep in a half sitting position. When an obese patient with a hollow back at his thoracic level is lying down, the weight is supported by the ribs of the right and the left side of the thorax and compresses the thorax to a smaller volume. At each inspiration more force is needed to rotate the ribs and to elevate the heavy thorax than when the right and left side of the thorax can move free.

This invention can elevate the spinal column at the upper and at the lower thoracic level and therefore the thorax is no more compressed and the ribs can move free. Inspiration requires less force and the patient can be breathing easier even when lying down. Obese and non-obese patients can benefit from this support system according to the hollow structure of their back. This is the second way to apply this invention.

In this invention the upper and lower spinal column elevation system can also be inflated in a synchronized way with the respiration of the patient. During inspiration the spinal column is elevated increasing the lung capacity, increasing the thoracic compliance and facilitating the inspiration. The ribs rotate due to loss of weight comparable with the action of the inspiratory muscles of the thoracic wall. During expiration the elevation is lowered, compressing the thorax, lowering the compliance and therefore facilitating the expiration. The ribs rotate back opposite to the action of the inspiratory muscles. The work of breathing is reduced for the patient resulting in larger minute volume ventilation or less oxygen consumption.

The support system cannot be used in a normal bed unless the mattress and mattress support is harder and does not allow the body to sink down. This is the third way to apply this invention. In paralyzed patients repetitive inflation and deflation of the support system creates a small inspiration and expiration, however not sufficient, as the diaphragm function does not act and move even in an opposite way.

### BRIEF DESCRIPTION OF THE DRAWINGS

In order to better understand the invention and to illustrate it in practice, non-limiting examples of some preferred embodiments will now be described, with reference to the accompanying drawings, in which:

FIG. 1 is a schematic illustration of the device when all balloons are inflated. FIG. 2 is a schematic illustration of the device when positioned under the patient without being inflated.

FIG. 3 is a schematic illustration of the device when positioned under the patient and being inflated for intubation.

FIG. 4 is a schematic illustration of the device when positioned under the patient and being inflated for spontaneous breathing.

FIG. 5 is a schematic illustration of the device when positioned under the patient and being inflated for central venous puncture

FIG. 6 is a schematic illustration of the device when positioned under the patient and being inflated for trendelenburg position during operation.

### DETAILED DESCRIPTION OF PREFERRED EMBODIMENTS

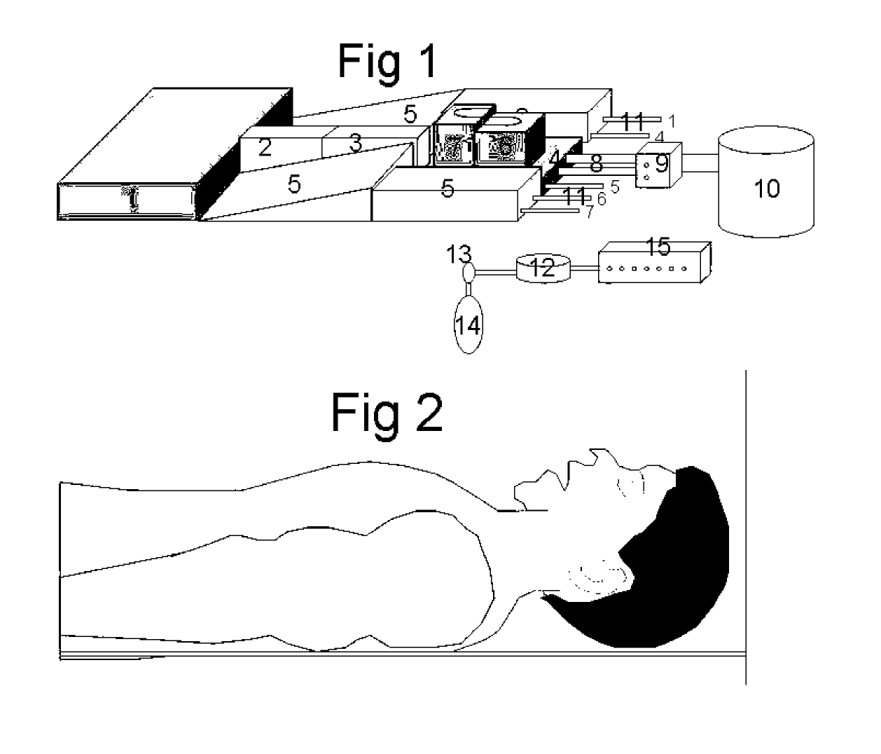

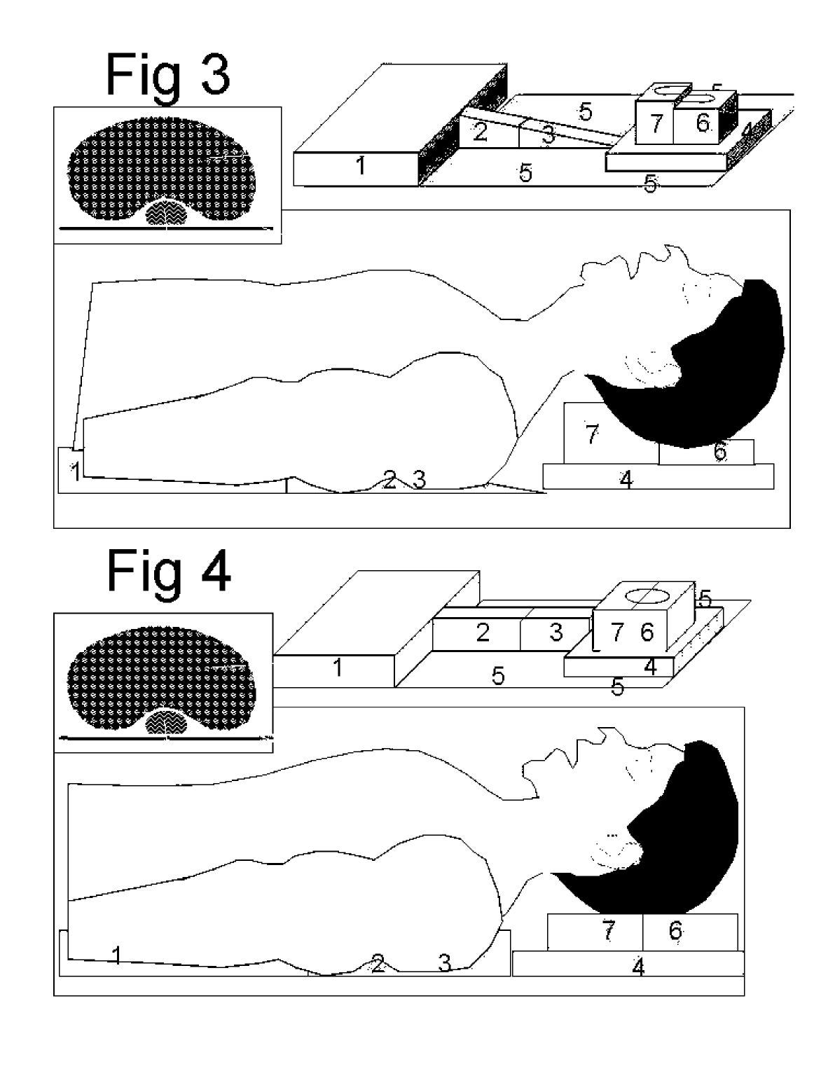

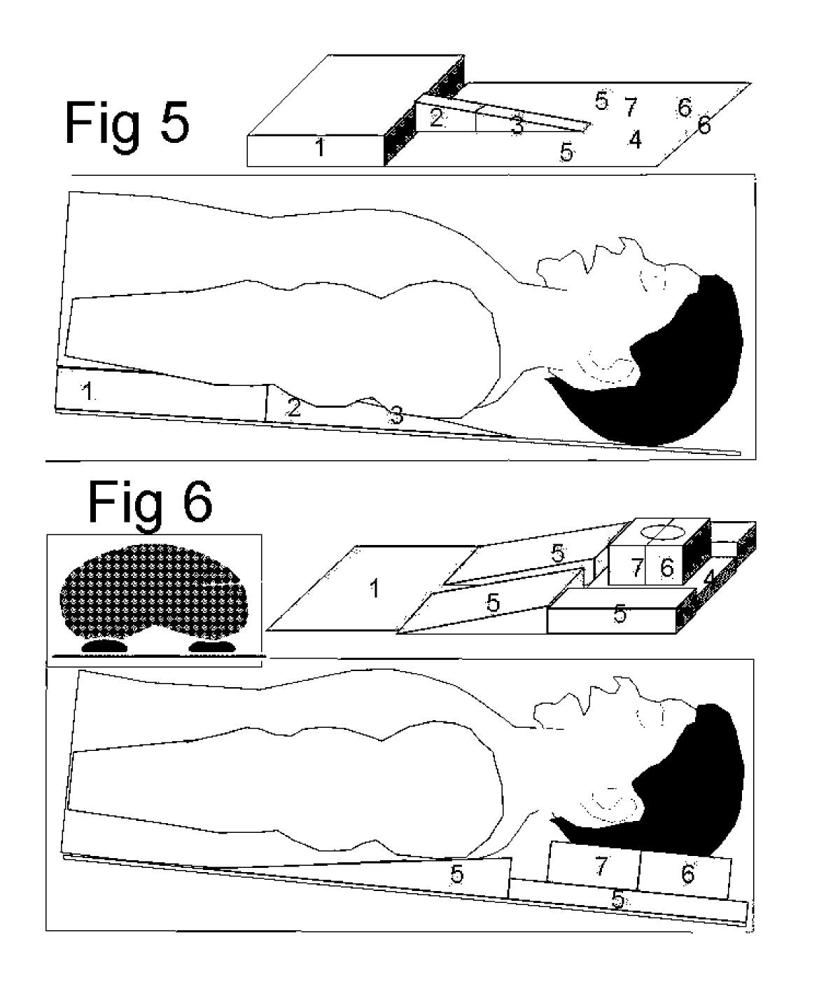

An intubation positioning, breathing facilitator and non-invasive assist ventilator device in accordance with a preferred embodiment of the present invention, is shown in FIG. 1 where all the balloons of the device are inflated. Number 1 is a flat balloon pillow positioned under the abdomen. Number 2 is a tube like balloon under the lower thoracic spinal column. Number 3 is the balloon positioned under the upper thoracic spinal column needed to inclinate the trachea. Number 4 is a triangular balloon on the right and on the left side positioned under the shoulders. Number 5 is a flat balloon pillow positioned under the head. Number 6 is a half circular balloon positioned under the upper head to elevate the head. Number 7 is a second half circular balloon positioned under the lower head to allow hyper extension of the head during intubation. Number 8 is a double large tube connection, one to balloon number 2 and one to balloon number 3 to allow rapid inflation and deflation when assisting the ventilation. Number 9 is a valve that allows closure of the connection tube number 8 when not in use and connection to the high-pressure ventilator 10 when in use. Number 11 are the connection tubes from all the other balloons number 1 to 7 to the inflation device. Number 12 is the manometer measuring the pressure inside the different balloons. Number 13 is the three-way valve to release air from each balloon. Number 14 is a manual air pump with a one-way valve to inflate the balloon. Number 15 the control system connecting each balloon independently to the manometer and the inflation device. FIG. 1 is a situation that is normally not used. In FIG. 2 no balloon is inflated creating a flat mat under the patient. In FIG. 3 balloons 1, 2, 4, 6 and 7 are inflated. Balloon 3 might be inflated if more anterior movement of the trachea is needed than inclination of the trachea to the pharyngeal line. Balloon 7 is more inflated than balloon 6 creating hyperextension of the head. Balloon 4 is inflated to flex the cervical column and inclinate the pharynx towards the tracheal line. A cross section of the thorax shows also the elevation of the right and left thoracic wall. In FIG. 4 all balloons are inflated except number 5. Balloon 3 is equally inflated as balloon 2 lifting up the total thoracic spinal column. Balloon 6 is equally inflated as balloon 7 preventing hyper extension. The same thoracic elevation is seen as in Fig 3. In FIG. 5 only balloon 1 and 2 is fully inflated. In this position the table is also inclinated lowering the head to facilitate central venous puncture of the vena jugularis. Balloon 3 might be also inflated to elevate the central thorax and lower the shoulders, facilitating access to the vena subclavia. In FIG. 6 the table might get even a more Trendelenburg inclination and inflating balloons 4, 5, 6 and 7 will prevent the patient from gliding of the table. This is safer than a shoulder bloc that might cause nerve lesions. On the cross section through the thorax the right and left support are seen.

### REFERENCES

1: Barnas G M, Green M D, Mackenzie C F, Fletcher S J, Campbell D N, Runcie C, Broderick G E.
Effect of posture on lung and regional chest wall mechanics.
Anesthesiology. 1993 February; 78(2):251-9.
2: Baydur A, Behrakis P K, Zin W A, Jaeger M J, Weiner J M, Milic-Emili J.
Effect of posture on ventilation and breathing pattern during room air breathing at rest.
Lung. 1987;1 65(6):341-51. Erratum in: Lung 1 988;1 66(3):1 88.
3: Behrakis P K, Baydur A, Jaeger M J, Milic-Emili J.
Lung mechanics in sitting and horizontal body positions.
Chest. 1983 April; 83(4):643-6.
4: Brander P E., Mortimore I L., Douglas N J.
Effect of obesity and erect/supine posture on lateral cephalometry: relationship to sleep-disordered breathing.
Eur Respir J. 1999 February; 13(2):398-402.
5: Isono S, Tanaka A, Ishikawa T, Tagaito Y, Nishino T.
Sniffing position improves pharyngeal airway patency in anesthetized patients with obstructive sleep apnea.
Anesthesiology. 2005 September; 103(3):489-94.
6: Isono S, Tanaka A, Tagaito Y, Ishikawa T, Nishino T.
Influences of head positions and bite opening on collapsibility of the passive pharynx.
J Appi Physiol. 2004 July; 97(1):339-46. Epub 2004 March 12.
7: Jan M A, Marshall I, Douglas N J.
Effect of posture on upper airway dimensions in normal human.
Am J Respir Crit Care Med. 1994 January; 149(1):145-8.
8: Lumb A B, Nunn J F.
Respiratory function and ribcage contribution to ventilation in body positions commonly used during anesthesia.
Anesth Analg. 1991 October; 73(4):422-6.
9: Martin S E, Mathur R, Marshall I, Douglas N J.
The effect of age, sex, obesity and posture on upper airway size.
Eur Respir J. 1997 September; 10(9):2087-90.
10: Michels A, Decoster K, Derde L, Vleurinck C, Van de Woestijne K P.
Influence of posture on lung volumes and impedance of respiratory system in healthy smokers and nonsmokers.
J AppI Physiol. 1991 July; 71 (1):294-9.
11: Ogunnaike B O, Jones S B, Jones D B, Provost D, Whitten C W.
Anesthetic considerations for bariatric surgery.
Anesth Analg. 2002 December; 95(6):1793-805.
12: Pae E K, Lowe A A, Sasaki K, Price C, Tsuchiya M, Fleetham J A.
A cephalometric and electromyographic study of upper airway structures in the upright and supine positions.
Am J Orthod Dentofacial Orthop. 1994 July; 106(1):52-9.
13: Sasaki H, Hida W, Takishima T.
Influence of body position on dynamic compliance in young subjects.
J App Physiol. 1977 May; 42(5):706-10.
14: Vitacca M, Clini E, Spassini W, Scaglia L, Negrini P, Quadri A.
Does the supine position worsen respiratory function in elderly subjects?
Gerontology. 1996;42(1)46-53.
15: Adnet F, Baillard C, Borron S W, Denantes C, Lefebvre L, Galinski M, Martinez C, Cupa M, Lapostolle F.
Randomized study comparing the “sniffing position” with simple head extension for laryngoscopic view in elective surgery patients.
Anesthesiology. 2001 October; 95(4):836-41.
16: Schmitt H J, Mang H.
Head and neck elevation beyond the sniffing position improves laryngeal view in cases of difficult direct laryngoscopy.
J Clin Anesth. 2002 August; 14(5):335-8.
17: Adnet F, Borron S W, Dumas J L, Lapostolle F, Cupa M, Lapandry C. Study of the “sniffing position” by magnetic resonance imaging.
Anesthesiology. 2001 January; 94(1):83-6.
18: Hochman I I, Zeitels S M, Heaton J T.
Analysis of the forces and position required for direct laryngoscopic exposure of the anterior vocal folds.
Ann Otol Rhinol Laryngol. 1999 August; 108(8):71 5-24. Review.
19: Collins J S, Lemmens H J, Brodsky J B, Brock-Utne J G, Levitan R M.
Laryngoscopy and morbid obesity: a comparison of the “sniff” and “ramped” positions.
Obes Surg. 2004 October; 14(9):1171-5.
20: Hastings R H, Hon E D, Nghiem C, Wahrenbrock E A.
Force and torque vary between laryngoscopists and laryngoscope blades.
Anesth Analg. 1996 March; 82(3):462-8.
21: Tamura M, Ishikawa T, Kato R, Isono S, Nishino T.
Mandibular advancement improves the laryngeal view during direct laryngoscopy performed by inexperienced physicians.
Anesthesiology. 2004 March; 100(3):598-601.
22: Krantz M A, Poulos J G, Chaouki K, Adamek P.
The laryngeal lift: a method to facilitate endotracheal intubation.
J Clin Anesth. 1993 July-August; 5(4):297-301.
23: Levitan R M, Mechem C C, Ochroch E A, Shofer F S, Hollander J E.
Head-elevated laryngoscopy position: improving laryngeal exposure during laryngoscopy by increasing head elevation.
Ann Emerg Med. 2003 March; 41(3):322-30.

What is claimed is:

1. An intubation support, breathing facilitator and non-invasive assist ventilator apparatus
that first supports, elevates and tilt the spinal column and the head for aiding in aligning an individual's oral, pharyngeal, laryngeal and tracheal axes for facilitating the endotracheal intubation during anaesthesia induction,
that secondly supports and elevates the spinal column for aiding the patient in breathing spontaneously and that thirdly elevates the spinal column repetitively in synchrony with the spontaneous ventilation and thereby assist the ventilation in a non-invasive way.
An intubation support, breathing facilitator and non-invasive assist ventilator, comprising two independent gradual inflatable balloons,
the first balloon positioned under the thoracic spinal column and the second balloon positioned under the head,
a pressure monitorthat allows to read the pressure inside the two balloons independently,
a manual or an electric air pump and controller that allows to inflate the two balloons independently,
a respiratory monitor that allows to measure the beginning of a spontaneous inspiration and expiration and
a large tube connection on the first balloon that supports the spinal column and is connected to
a high pressure ventilator that can be triggered by the respiratory monitor or works independently at a fixed frequency and volume or pressure.

2. The intubation support, breathing facilitator and non-invasive assist ventilator apparatus of claim 1, wherein said first balloon consists of a cylindrical tube device that supports only the spinal column and keeps the right and left dorsal thorax free from the table, wherein said second balloon is a circular tube around the head that stabilises and elevates the head.

3. The intubation support, breathing facilitator and non-invasive assist ventilator apparatus of claim 2 wherein the first said balloon under the spinal column is made of two independent compartments, one under the abdominal and lower thoracic spinal column and one under the upper thoracic spinal column, allowing to be inflated differently and thereby rotating the axis of the trachea more in line with the axis of the pharynx.

4. The intubation support, breathing facilitator and non-invasive assist ventilator apparatus of claim 3 wherein the balloon under the spinal column consists of more than two balloons allowing to be inflated differently and thereby flexing and extending the spinal column at every possible point.

5. The intubation support, breathing facilitator and non-invasive assist ventilator apparatus of claim 4 wherein the said balloons can be inflated with air or fluid, thereby allowing adaptation of the amount of support and allowing inflating and supporting only when needed. The said apparatus can be positioned as a flat device under the patient before anaesthesia induction and does not need to be removed during or direct after surgery.

6. The intubation support, breathing facilitator and non-invasive assist ventilator apparatus of claim 5, wherein said second balloon is circular and is made of two independent compartments, one under the lower part of the head and one under the upper part of the head allowing gradual hyperextension and gradual elevation of the head. When both balloons are inflated at the same pressure the head will only elevates. When said compartment under the lower part of the head is more inflate than the compartment under the upper head, the head will hyperextend. Opposite inflation will flex the head.

7. The intubation support, breathing facilitator and non-invasive assist ventilator apparatus of claim 6 wherein the said balloons are fixed together as one unit on a layer fixed in place with weak glue that attaches to the skin of the patient and to the operating table. In a bed a plate of hard material that does not fold like wood, plastic or metal is placed between the inflatable apparatus and the bed to prevent the right or left side of the thorax to rest on the bed.

8. The intubation support, breathing facilitator and non-invasive assist ventilator apparatus of claim 7, wherein said balloons are made from polyvinyl chloride or other plastics that allows being disposable.

9. The intubation support, breathing facilitator and non-invasive assist ventilator apparatus of claim 8, wherein only the balloons under the abdomen and under the thoracic spinal column are inflated to facilitate central venous puncture of the vena juguralis by creating an inclination of the patient and of the vena subclavia by elevating the central part of the thorax and lowering the shoulders.

10. The intubation support, breathing facilitator and non-invasive assist ventilator apparatus of claim 9, further comprising a comfort layer disposed on the upper surface of the different balloons that provides physical comfort to the patient's skin.

11. The intubation support, breathing facilitator and non-invasive assist ventilator apparatus of claim 10, further comprising a self expanding foam inside one or more balloons to expand thereby causing inflation of said pressure chamber and a suction device to deflate the balloons.

12. The intubation support, breathing facilitator and non-invasive assist ventilator apparatus of claim 11, wherein the said inflatable balloons exist in different sizes, lengths and diameters according to the age and size of the patient.

13. The breathing facilitator apparatus of claim 12, wherein said balloons under the spinal column are inflated only to a fixed volume and pressure and the patient might use a normal comfortable pillow instead of said balloon two and three.

14. The breathing facilitator apparatus of claim 12, wherein the patient is breathing spontaneous but can be from fully awake till unconscious under deep anaesthesia.

15. The breathing facilitator apparatus of claim 12, wherein the patient is not breathing spontaneous but is invasive or non-invasive ventilated by a standard ventilator in a volume controlled, pressure controlled or other ventilation mode and thereby requires lower airway pressures or allows higher tidal volumes when the said breathing facilitator apparatus is used compared to a ventilation without the use of the breathing facilitator apparatus.

16. The non-invasive assist ventilator apparatus of claim 12, wherein the pressure or the volume in the said balloon under the spinal column is changed from a minimum to a maximum value at the beginning of the inspiration and is changed from a maximum to a minimum value at the beginning of the expiration.

17. The non-invasive assist ventilator apparatus of claim 12, wherein a ventilator with compressed air is used that can deliver tidal volumes up to 5 litre and pressures up to 300 cmH2O at a frequency up to 0,5 Hz, using the said large tube connection on the said balloon under the spinal column. The temperature of the air is regulated to achieve a temperature of 37 degree Celsius.

18. The intubation support, breathing facilitator and non-invasive assist ventilator apparatus of claim 12, wherein the said inflatable balloons are incorporated in a triangular shaped pillow under the shoulders that is also inflatable and that gradual elevates them and prevent gliding of the patient on the operating table during trendelenburg position for central venous puncture or operation.

19. The intubation support, breathing facilitator and non-invasive assist ventilator apparatus of claim 18, wherein the said inflatable balloons and pillows are divided in two or more parts that allow alternating inflation of one half of them during the first period and the other half during the second period to prevent decubitus hypo perfusion.

20. The intubation support, breathing facilitator and non-invasive assist ventilator apparatus of claim 18, wherein the said alternating inflatable balloons and pillows are inflated with warm air, regulated at a temperature up to 43° Celsius to allow warming up of the patient.
